(19) United States
(12) Patent Application Publication
(10) Pub. No.: US 2009/0076485 A1
(43) Pub. Date: Mar. 19, 2009

(54) Alarm system for implantable pumps for intravenous drug delivery

(76) Inventor: Kamal K. Mubarak, Gainesville, FL (US)

Correspondence Address: Rohm & Monsanto, PLC, 12 Rathbone Place, Grosse Pointe, MI, 48230, US

(21) Appl. No.: 12/291,866

(22) Filed: Nov. 13, 2008

Related U.S. Application Data
(63) Continuation-in-part of application No. 11/414,734, filed on Apr. 28, 2006, now abandoned.
(60) Provisional application No. 60/676,378, filed on Apr. 29, 2005.

(51) Int. Cl. A61M 5/31; A61M 25/16; A61M 5/20

(52) U.S. Cl. 604/533; 604/247; 604/151; 604/507

(57) ABSTRACT

An arrangement for administering a medication to a patient. The arrangement is provided with a dual output element having first and second outputs, and an input for receiving the medication. A first catheter is coupled to the first output of the dual output element for delivering the medication intravenously, the first catheter being provided with a pressure valve that opens at a pressure P₁. A second catheter is coupled to the second output of the dual output element for delivering the medication subcutaneously upon failure of the first catheter to deliver the medication intravenously, the second catheter being provided with a pressure valve that opens at a pressure P₂, where P₁>P₂. A monitored radiopaque disk valve arrangement changes its status at a pressure P₃ when a catheter occlusion is present, and is also used to obviate the need for two different types of catheters.

### CROSS-REFERENCE TO RELATED APPLICATIONS

This application is a continuation-in-part patent application of U.S. Ser. No. 11/414,734 filed on Apr. 28, 2006 in the name of the inventor herein, which is a continuation of, and claims the benefit of, U.S. Provisional Application Ser. No. 60/676,378 filed on Apr. 29, 2005, the disclosures of which are incorporated herein by reference.

### BACKGROUND OF THE INVENTION

1. Field of the Invention

This invention relates generally to systems and arrangements for delivering medication to a patient, and more particularly to a system that delivers medication intravenously to the patient, and subcutaneously to the patient when intravenous delivery fails.

2. Description of the Prior Art

Pulmonary arterial hypertension is a condition that causes high blood pressure in the lungs and leads to right heart failure. Untreated average survival is 2-3 years. This condition occurs in several varieties, and may be caused in different patients by congenital heart disease, HIV, liver disease, scleroderma/rheumatoid arthritis/SLE, etc.

Treatment of pulmonary arterial hypertension includes the administration of bosentan (Tracleer, an endothelin receptor antagonist that is taken twice a day by mouth), epoprostenol (Flolan, an intravenous prostaglandin analog given continuously), and treprostinil (Remodulin, an intravenous or subcutaneous prostaglandin analog), sildenafil (RevatioNiagra, a PDE5 inhibitor taken 3 times a day), ambrisentan (Letairis/olibris, an endothelin receptor antagonist taken once a day), iloprost (Ventavis, an inhaled prostaglandin analog taken 6-9 times a day), and sitaxsentan (Thelin, an endothelin receptor antagonist taken once a day, not approved in the United States so far). Tadalafil (Cialis, a PDE5 inhibitor taken once a day) may soon receive approval for treatment of pulmonary arterial hypertension. Intravenous prostaglandin analogs are more effective than oral or inhaled therapy, but are difficult to manage for the patient. Bosentan/Tracleer and ambrisentan/Letairis cost about $50,000 per year, sildenafil/Revatio/Viagra costs about $10,000 per year, epoprostenol/Flolan typically costs over $100,000 per year, iloprost/Ventavis costs about $80,000 per year, and Remodulin costs over $150,000 per year.

Intravenous therapy is reserved for those who are severely ill, and is very cumbersome for patients. Once the patient is connected to a pump, disconnection can lead to death. Medication has to be given without any interruptions whatsoever. Delivery usually requires a chronic indwelling (e.g., Hickman) catheter, which sometimes gives rise to sepsis (infection) and thrombosis (clots). Patients have to learn how to mix the medication. Epoprostenol/Flolan is unstable and the pump needs to be packed in ice.

One solution that has been proposed in the art is to use a stable prostaglandin analog that can be given like insulin with an insulin pump. This medication is called treprostinil/Remodulin, a medication that is stable (no ice pack), longer lasting (disconnection is less likely to be fatal), and can be administered with a smaller pump. However, although treprostinil/Remodulin works well, it causes a lot of pain where it is injected. Therefore, very few patients remain on the therapy long term.

Another proposed solution, since subcutaneous (under the skin) infusion with an insulin pump causes pain, is to administer the medication with an implantable pump. One such implantable pump is manufactured by Medtronic (Minneapolis, Minn.) called the “SynchroMed Implantable Pump.” Using such a pump, the medication can be delivered intravenously, continuously, and painlessly. The patient would require a pump refill every 3 to 6 months. It is a problem with the SynchroMed Implantable pump, however, that it does not have an alarm to alert the patient that no medication is being delivered. If the intravenous line occludes (clots), the patient can become severely ill, and perhaps die.

There are two kinds of implantable pumps on the market for intravenous delivery of drugs. The first kind is a constant flow variety that delivers only a factory preset rate of the drug. Changing the concentration of the drug can change the dose of the drug. The second kind of pump is the programmable variety that can be programmed to deliver drug at variable rates with an external controller. The patient and/or the healthcare professional can also deliver boluses of drug.

Constant flow rate pumps usually contain a gas, or a spring, that is compressed when the drug is injected into the pump. The compressed gas, or spring, provides the energy required to deliver the drug to the patient. Programmable pumps have a battery and sophisticated electronics for controlling the delivery rate of drug to the patient. These programmable pumps are similar to pacemakers, and as previously noted, some pacemaker manufactures, such as Medtronic, Inc., manufacture such devices. U.S. Pat. No. 6,537,268 is illustrative of a programmable pump of the type which is useful in the practice of the invention, and the disclosure therein is incorporated herein by reference.

As noted, however, the implantable pumps that presently exist in the marketplace do not have a no-delivery alarm that would inform a patient that the implantable pump has stopped working. On the other hand, almost all external intravenous or subcutaneous pumps have no-delivery alarms. For pain medications, antibiotics, or chemotherapy, failure of delivery can have adverse consequences, but these consequences are not immediately fatal. Therefore, implantable pumps have been used for such drugs. For certain drugs, however, such as the prostaglandin analogs useful in treating pulmonary arterial hypertension, sudden cessation of drug delivery can be fatal.

Antibiotics and pain medications are typically not delivered over an extended period of time, and therefore catheter occlusion is a relatively rare occurrence. Prostaglandin analogs, on the other hand, must be continuously administered once therapy is initiated. Therefore, prostaglandin analog delivery for pulmonary arterial hypertension has so far only been performed with external pumps. To adapt implantable pumps for delivery of prostaglandin analogs, a no-delivery alarm, as well as a backup delivery system is needed.

Previously, the prostaglandin analogs on the market were not stable at body temperature nor were they available in high enough concentrations. However, due to recent technological developments, highly stable and highly concentrated prostaglandin analogs are available, and hence, would be good candidates for delivery via implantable pumps. Nevertheless, intravenous delivery of prostaglandin analogs by an implantable pump in a system that does not have a no-delivery alarm would make drug delivery for pulmonary arterial hypertension highly risky. As indicated above, prostaglandin analog delivery must not be interrupted. Moreover, since prostaglandin analog delivery has to be given for the remainder of the patient's life, often more than 5 or 10 years, occlusion of the catheter is a very real possibility.

There is, therefore, a need for an implantable drug delivery pump that incorporates an alarm to alert the patient and/or a health care professional, and that, preferably additionally actuates a backup drug delivery system.

### SUMMARY OF THE INVENTION

The foregoing and other objects are achieved by this invention, which provides an arrangement for administering a medication to a patient. In accordance with the invention, the arrangement is provided with a dual output element having first and second outputs, and an input for receiving the medication. A first catheter is coupled to the first output of the dual output element for delivering the medication intravenously to the patient. Additionally, a second catheter is coupled to the second output of the dual output element for delivering the medication subcutaneously to the patient upon failure of the first catheter to deliver the medication intravenously.

In one embodiment of the invention, the first catheter is provided with a pressure valve that opens at a pressure P₁. Additionally, the second catheter is provided with a pressure valve that opens at a pressure P₂, where P₁>P₂.

In a highly advantageous embodiment of the invention, there is further provided a disk valve arrangement disposed within the dual output element. The disk valve arrangement is configured to open at pressure P₃, where P₂>P₃>P₁. In a further embodiment, the disk valve arrangement is formed of a radiopaque material.

Further in accordance with the invention, the dual output element is provided with a pressure-sensitive valve arrangement at the input. In a specific illustrative embodiment of the invention, the pressure-sensitive valve arrangement is provided with a stopper member that is axially translatable in the vicinity of an input port. Additionally, a resilient biasing element is arranged in communication with the stopper member.

An implantable pump delivers the medication to the input of the dual output element.

In accordance with a further apparatus aspect of the invention, there is provided an arrangement for administering a medication to a patient. The arrangement is provided with a dual output element having first and second outputs, and an input for receiving the medication. A first catheter is coupled to the first output of the dual output element for delivering the medication intravenously, the first catheter being provided with a pressure valve that opens at a pressure P₁. Additionally, a second catheter is coupled to the second output of the dual output element for delivering the medication subcutaneously upon failure of the first catheter to deliver the medication intravenously, the second catheter being provided with a pressure valve that opens at a pressure P₂, where P₁>P₂.

In one embodiment of this further apparatus aspect of the invention, there is provided a pressure-sensitive valve arrangement disposed at the input of the dual output element. The pressure-sensitive valve arrangement is provided, in a specific illustrative embodiment of the invention, with a stopper member that is axially translatable in the vicinity of an input port. Additionally, a resilient biasing element is arranged in communication with the stopper member.

In one embodiment, there is further provided a disk valve arrangement disposed within the dual output element. The disk valve arrangement is configured to open at a pressure P₃. As previously noted, the disk valve arrangement is, in some embodiments, formed of a radiopaque material.

There is additionally provided in some embodiments of the invention an implantable pump for being implanted into the patient and for delivering the medication to the input of the dual output element.

In accordance with a method aspect of the invention, there are provided the steps of:

first delivering the medication intravenously to the patient; and

second delivering the medication subcutaneously to the patient automatically upon failure of the step of first delivering.

In one embodiment of this method aspect of the invention, prior to performing the step of first delivering, there is provided the step of introducing the medication from an infusion pump to a pressure-sensitive valve. The step of first delivering is performed at a pressure at least P₁. Additionally, the step of second delivering is performed at a pressure P₂, where P₁>P₂.

In an advantageous embodiment of the method aspect of the invention, there is provided the step of monitoring the status of a disk valve arrangement. In some embodiments of the invention the step of monitoring is effected using standard imaging technologies on a disk valve arrangement formed of a radiopaque material. The status of the disk valve arrangement will reveal the presence of one or more occlusions in the catheters, or that the catheters have incorrectly been installed in the patient. More specifically, if during surgical implantation the operator incorrectly places the subcutaneous catheter into the intravascular space and the intravascular catheter into the subcutaneous space, the disk valve accommodates the error. In fact, with the use of a radiopaque disk valve that opens at a higher pressure than the catheter tip valves, the need for two different types of catheters (intravenous and subcutaneous) is eliminated, i.e., P₃>P₂=P₁.

The present invention contemplates the design of an implantable pump alarm that is to be implanted in the subcutaneous space in the abdomen, amongst other places in the body. The system, as noted, uses a T- or Y-connector with two catheters extending therefrom. One catheter is inserted into a vein to deliver the medication. The other catheter, which has a pop-off valve, remains in the subcutaneous space. If the intravenous catheter occludes, the subcutaneous catheter delivers the medication. This delivery through the subcutaneous catheter causes pain, and the pain constitutes the alarm to the patient. The patient does not experience ill effects because he or she continues to receive the medication when the catheter in the vein occludes.

These and other objects, features, and advantages, are accomplished in the drug delivery system of the present invention that, in a preferred embodiment, delivers highly concentrated, stable prostaglandin analogs via a programmable implantable pump. The programmable implantable pump, which may be of any variety, such as the SynchroMed EL brand programmable pump marketed by Medtronic, Inc., is provided with two drug delivery catheters. One of the catheters will be placed intravenously and will constitute the primary route of delivery of the drug. The second catheter will be placed in a subcutaneous space at a small distance from the subcutaneous implantation site of the pump.

At least one of these catheters, and preferably both catheters, will have a valve, illustratively a slit valve, at its drug delivery tip. In the case where slit valves are used in both catheters, the slit valve at the drug delivery tip of the intravenous catheter will open at a pressure, P₁, which is the critical opening pressure of the intravenous catheter. The subcutaneous catheter has a slit valve at its drug delivery tip that only opens at pressure, P₂, and delivers drug if the pressure at its tip is higher than the critical opening pressure of the intravenous catheter (i.e., P₂>P₁). As is known in the art, catheters typically comprise an elongated hollow polymeric tube having a hollow tip at a distal end for delivering fluid drug to the patient. An example of a catheter, suitable for use in the practice of the invention includes, without limitation, the Groshong PICC catheter manufactured by Bard Access (New Providence, N.J.).

Both of the catheters are connected to the implantable pump via a T- or a Y-connector. The pump will deliver the drug preferentially to the intravascular space, which is the preferred route of administration. If there is an occlusion of the intravenous catheter, or a thrombosis of the vein, the slit valve at the end of the subcutaneous catheter will open and the drug will be delivered subcutaneously.

This drug delivery system is particularly useful for the administration of prostaglandin analogs. Prostaglandin analogs have a characteristic that is peculiar to them. While intravenous delivery can cause jaw pain and other symptoms, subcutaneous delivery causes pain at the injection site. This is one reason for the lack of popularity of subcutaneous delivery of prostaglandin analogs, despite other advantages of this route, including less risk of infection and thrombosis. This property of prostaglandin analogs, which is the “alarm,” works to the patient's advantage in the no-delivery alarm system of the present invention.

Since subcutaneous and intravenous delivery of prostaglandin analogs have 100% bioavailability, and have been shown to be bioequivalent, there will be no interruption in drug delivery, and consequently no adverse effects. Subcutaneous delivery will eventually cause pain at the tip of the subcutaneous catheter leading to the patient and physician becoming aware of the problem. The catheter system can then be replaced as an elective procedure rather than as an emergency.

Of course, in the case of drugs that do not cause pain when injected subcutaneously, or the minority of patients who do not experience pain with prostaglandin analogs, the so-called “alarm” would be ineffective. However, the backup drug delivery system of the present invention would still be useful to continue drug delivery if the intravenous catheter occludes. X-ray surveillance can detect the position of the radiopaque disk valve indicating whether the drug is being delivered via the primary catheter or via the backup catheter (necessitating replacement).

### BRIEF DESCRIPTION OF THE DRAWING

Comprehension of the invention is facilitated by reading the following detailed description, in conjunction with the annexed drawing, in which:

FIG. 1 is a simplified schematic representation of an implantable pump for intravenous drug delivery constructed in accordance with the present invention; and

FIG. 2 is a simplified schematic representation of a specific illustrative embodiment of the implantable pump for intravenous drug delivery wherein there is additionally provided a disk valve that enables detection of occlusion without surgical intervention using x-ray surveillance.

### DETAILED DESCRIPTION

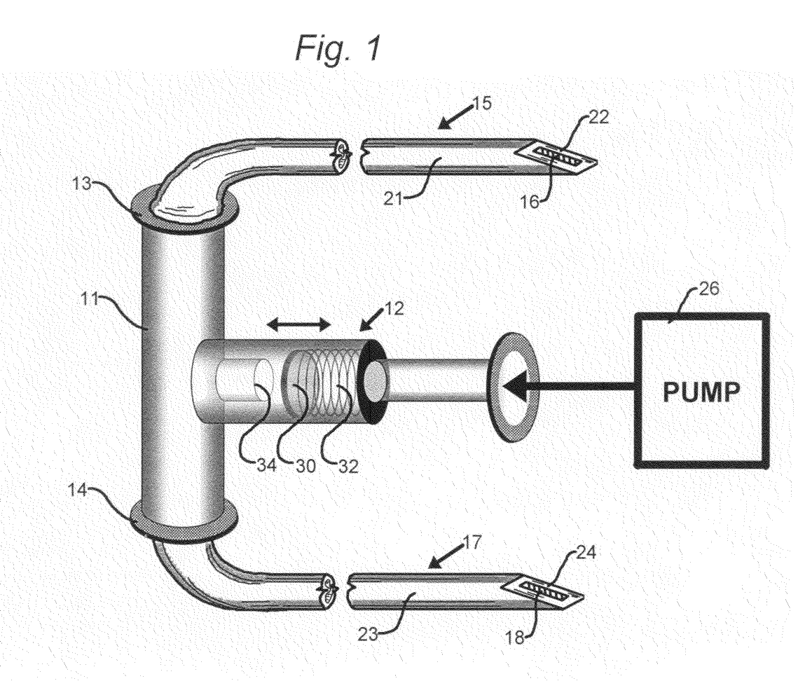

FIG. 1 is a simplified schematic representation of a portion of drug delivery system of the present invention comprising a T-connector 11 that receives fluid drug from an implantable infusion pump 26. The drug is delivered via a conduit, such as a hollow polymeric tube, from the outlet end of pump 26 to T-connector 11 via a pressure sensitive valve 12 and exits the T-connector either through intravenous catheter end 13 or subcutaneous catheter end 14.

T-connector 11 is shown to have a pressure-responsive arrangement in the form of a pressure-sensitive valve 12. In other embodiments, T-connector 11 may be a Y-connector element (not shown). As shown, pressure-sensitive valve 12 is provided with a stopper 30 that is resiliently biased against a spring 32. The stopper is disposed in the vicinity of an input port 34 within T-connector 11.

Intravenous catheter end 13 connects to a first catheter 15 that constitutes a hollow tubular body 21 and a hollow tip 22 for intravenous placement in a patient (not shown). Subcutaneous catheter end 14 connects to a second catheter 17 that constitutes a hollow tubular body 23 and a hollow tip 24 for subcutaneous placement in the same patient.

The distal end of the intravenous catheter that is connected to T-connector 11 at intravenous catheter end 13, has, in preferred embodiments, a slit valve 16 in the drug delivery tip 22 that opens at a pressure, P₁, which is the critical opening pressure of intravenous catheter 15. The distal end of subcutaneous catheter 17 has a slit valve 18 at its drug delivery tip 24 that only opens at pressure, P₂, to deliver drug if the pressure at tip 24 is higher than the critical opening pressure of the intravenous catheter at tip 22 (i.e., P₂>P₁).

Although shown in simplified schematic form in the figure, implantable pump 26 typically includes an internal fluid chamber or reservoir (not shown) for receiving and storing a supply of a fluid drug; a pump mechanism (not shown); and programmable control system (not shown) for operating pump 26 mechanism to deliver discrete doses of the drug from the internal reservoir to the patient. The drug is typically delivered to the body through a catheter connected to an output port of pump 26. A refill port is typically provided on pump 26 to permit transcutaneous access via a needle for periodically refilling the internal reservoir with a fresh supply of drug.

In a method of use embodiment of the invention, the implantable infusion pump is installed subcutaneously in a patient. Catheter 15 is installed intravascularly. Catheter 17 is installed subcutaneously a small distance from the implanted pump. Drug from the internal reservoir of pump 26 is supplied to T-connector 11 through pressure-sensitive valve 12. In normal operation, the drug exits the T-connector through intravenous catheter 15. Should intravenous catheter 15 occlude, the drug exits the T-connector through subcutaneous catheter 17. In the case of delivery of prostaglandin analogs, a painful sensation will occur at the subcutaneous delivery site, thereby placing the patient on alert that the intravenous catheter is occluded and in need of replacement.

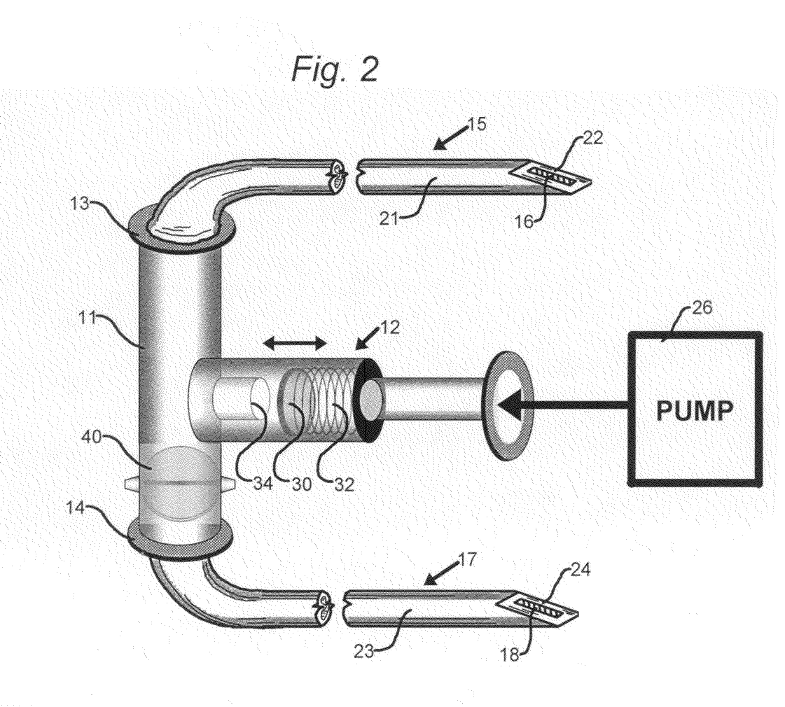

FIG. 2 is a simplified schematic representation of a specific illustrative embodiment of an implantable pump for intravenous drug delivery wherein there is provided a disk valve that enables detection of occlusion without surgical intervention. Elements of structure that have previously been discussed are similarly designated. As shown in this figure, there is incorporated within T-connector 11 a disk valve 40 (shown in phantom representation) that is, in this specific illustrative embodiment of the invention, made of a radiopaque material. Disk valve 40 is configured to open at pressure P₃, where P₂>P₃>P₁. When an occlusion event occurs, disk valve 40 changes position, and such change in position is readily detectable by conventional x-ray imaging, fluoroscopy, CT scanning, or other imaging modalities.

In the practice of some embodiments of the invention, a small quantity of radiopaque or radioactive material is injected into the pump. Subsequent x-ray or nuclear imaging can then be used for easy detection of the direction of flow.

In some embodiments of the invention, catheter valves (not shown) are placed at the ends of intravenous catheter 15 and subcutaneous catheter 17. These valves are useful to prevent reflux of blood or subcutaneous fluid (not shown) into the catheter tubing. Reflux can be minimized by reducing the catheter diameter to a sufficiently small gauge. Additionally, the catheter valves can be placed more proximally, and in some embodiments of the invention, the valves are included as part of the T connector.

The use of a disk valve, such as disk valve 40, provides the further advantage that if during surgical implantation the operator makes a mistake and places the subcutaneous catheter into the intravascular space and the intravascular catheter into the subcutaneous space, disk valve accommodates the error. More specifically, with the use of a radiopaque disk valve that opens at a higher pressure than the catheter tip valves, illustratively slit valves 16 and 18, the need for having two different catheters is eliminated. In such a design, P₁ can equal P₂, as long as P₃ is greater than both (i.e. P₃>P₂=P₁).

Although the invention has been described in terms of specific embodiments and applications, persons skilled in the art can, in light of this teaching, generate additional embodiments without exceeding the scope or departing from the spirit of the claimed invention. Accordingly, it is to be understood that the drawing and description in this disclosure are proffered to facilitate comprehension of the invention, and should not be construed to limit the scope thereof.

What is claimed is:

1. An arrangement for administering a medication to a patient, the arrangement comprising:
a dual output element having first and second outputs, and an input for receiving the medication;
a first catheter coupled to the first output of said dual output element for delivering the medication intravenously; and
a second catheter coupled to the second output of said dual output element for delivering the medication subcutaneously upon failure of said first catheter to deliver the medication intravenously.

2. The arrangement of claim 1, wherein said first catheter is provided with a pressure valve that opens at a pressure P₁.

3. The arrangement of claim 2, wherein said second catheter is provided with a pressure valve that opens at a pressure P₂, where P₂>P₁.

4. The apparatus of claim 3, wherein there is further provided a disk valve arrangement disposed within said dual output element, said disk valve arrangement being configured to open at pressure P₃, where P₂>P₃>P₁.

5. The apparatus of claim 4, wherein said disk valve arrangement is formed of a radiopaque material.

6. The apparatus of claim 2, wherein said second catheter is provided with a pressure valve that opens at a pressure P₂, where P₂=P₁, and wherein there is further provided a disk valve arrangement disposed within said dual output element, said disk valve arrangement being configured to open at pressure P₃, wherein P₃>P₂=P₁.

7. The arrangement of claim 1, wherein said dual output element is provided with a pressure-sensitive valve arrangement at the input.

8. The arrangement of claim 7, wherein said pressure-sensitive valve arrangement comprises:
a stopper member that is axially translatable in the vicinity of an input port; and
a resilient biasing element arranged in communication with said stopper member.

9. The arrangement of claim 1, wherein there is further provided an implantable pump for being implanted into the patient and for delivering the medication to the input of said dual output element.

10. An arrangement for administering a medication to a patient, the arrangement comprising:
a dual output element having first and second outputs, and an input for receiving the medication;
a first catheter coupled to the first output of said dual output element for delivering the medication intravenously, said first catheter being provided with a pressure valve that opens at a pressure P₁; and
a second catheter coupled to the second output of said dual output element for delivering the medication subcutaneously upon failure of said first catheter to deliver the medication intravenously, said second catheter being provided with a pressure valve that opens at a pressure P₂, where P₂>P₁.

11. The arrangement of claim 10, wherein said dual output element is provided with a pressure-sensitive valve arrangement at the input.

12. The arrangement of claim 11, wherein said pressure-sensitive valve arrangement comprises:
a stopper member that is axially translatable in the vicinity of an input port; and
a resilient biasing element arranged in communication with said stopper member.

13. The apparatus of claim 10, wherein there is further provided a disk valve arrangement disposed within said dual output element, said disk valve arrangement being configured to open at pressure P₃.

14. The apparatus of claim 13, wherein said disk valve arrangement is formed of a radiopaque material.

15. The arrangement of claim 10, wherein there is further provided an implantable pump for being implanted into the patient and for delivering the medication to the input of said dual output element.

16. A method of administering a medication to a patient, the method comprising the steps of:
first delivering the medication intravenously to the patient; and
second delivering the medication subcutaneously to the patient automatically upon failure of said step of first delivering.

17. The method of claim 16, wherein prior to performing said step of first delivering there is provided the step of introducing the medication from an infusion pump to a pressure-sensitive valve.

18. The method of claim 16, wherein said step of first delivering is performed at an opening pressure P₃.

19. The method of claim 18, wherein said step of second delivering is performed at a pressure P₂, where P₂>P₁.

20. The method of claim 16, wherein there is further provided the step of monitoring the status of a disk valve arrangement.
